(19) United States
(12) Patent Application Publication
(10) Pub. No.: US 2003/0212374 A1
(43) Pub. Date: Nov. 13, 2003

(54) Nose vacuum

(76) Inventor: Timothy Gusler, Pequannock, NJ (US)

Correspondence Address: Mr. Walter J. Tencza Jr., Suite 3, 10 Station Place, Metuchen, NJ, 08840, US

(21) Appl. No.: 10/141,210

(22) Filed: May 8, 2002

(51) Int. Cl. A61M 1/00

(52) U.S. Cl. 604/326

(57) ABSTRACT

A method and apparatus for vacuuming mucus from a nostril of a nose of a person is disclosed. The apparatus may be comprised of a vacuum device and a first tube. When the vacuum device is powered a suction force is created which causes mucus to be sucked out of the nostril of the nose of the person through the first tube. The first tube may have first and second ends. The first end of the first tube may be attached to a nozzle which can be inserted into the nostril while the second end of the first tube may be connected to the vacuum device. The apparatus may be further comprised of a second tube having a first end connected to a vacuum device and a second end connected to a receptacle, into which mucus is deposited. The second end of the first tube may also be connected to the receptacle and connected to the vacuum device through the receptacle.

### FIELD OF THE INVENTION

This invention relates to improved methods and apparatus concerning sucking mucus or other discharges from a small child's or infant's nose or generally from a person's nose or from an animal's nose.

### BACKGROUND OF THE INVENTION

Typically in the prior art a rubber ball attached to a stiff plastic tube is used to suck mucus or other discharges from a small child's or infant's nose.

### SUMMARY OF THE INVENTION

The present invention discloses a method and apparatus for vacuuming mucus from a nostril of a nose of a person. In one embodiment an apparatus is provided comprising a vacuum device and a first tube. When the vacuum device is powered a suction force is created which causes mucus to be sucked out of the nostril of the nose of the person through the first tube.

The first tube may have first and second ends. The first end of the first tube may be attached to a nozzle which can be inserted into a nostril while the second end of the first tube may be connected to the vacuum device. The apparatus may be further comprised of a second tube having a first end connected to a vacuum device and a second end connected to a receptacle. The second end of the first tube may also be connected to the receptacle and connected to the vacuum device through the receptacle.

The apparatus may also include an attachment device which attaches the receptacle to the vacuum device. A cover may be attached to the receptacle. The cover when placed on the receptacle may provide, in combination with the receptacle, a sealed airtight enclosure with the exception of a first and/or a second opening in the cover. The first tube may be inserted through the first opening and the second tube may be inserted through the second opening.

Third and fourth tubes may be provided which may be inserted through the first and second openings of the cover. The third tube may be connected to the first tube and the fourth tube may be connected to the second tube. The third tube and the fourth tubes may have each have first and second ends. The third tube may be fixed at a first location which above a bottom surface of the receptacle. The fourth tube may be fixed at a second location above a bottom surface of the receptacle. The first location may be lower than the second location to avoid deposited mucus from being sucked into the vacuum device itself.

The present invention includes a method comprising the steps of sucking mucus out from a nostril of a nose of a person by the use of a vacuum device. The method may be implemented which the components previously described.

The present invention in various embodiments can be useful for infants, small children, the handicapped, the elderly, for individuals generally, and for animals. The present invention can be used for persons in individuals' homes or in human hospitals, or may be used by veterinarians in animal hospitals, or by pet owners.

### BRIEF DESCRIPTION OF THE DRAWINGS

FIG. 1 shows an apparatus in accordance with an embodiment of the present invention; and

FIG. 2 shows an apparatus in accordance with a second embodiment of the present invention.

### DETAILED DESCRIPTION OF THE DRAWINGS

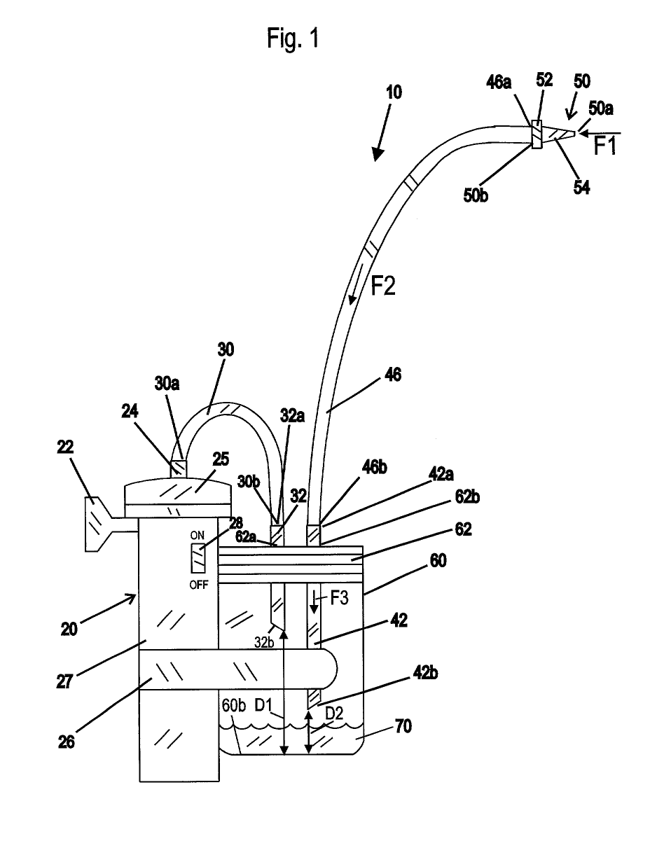

FIG. 1 shows an apparatus 10 in accordance with an embodiment of the present invention. Apparatus 10 is comprised of a vacuum device 20, a tube 30, a tube 32, a tube 42, a tube 46, a discharge receptacle 60, a top 62, and a nozzle 50. The vacuum device 20 is comprised of a vacuum discharge portion 22, a vacuum connection 24, an attachment device 26, a body portion 27, and an on-off switch 28.

The discharge jar 60 is attached to the vacuum device 20 by the attachment device 26. The attachment device 26 may be a clip. The tube 30 has a first end 30a which is attached to the vacuum connection 24 of the vacuum device. The tube 30 has a second end 30b which is attached to an end 32a of the tube 32. The tube 32 may be a stiff plastic tube which acts as an air line. The tube 32 may be inserted through a hole 62a in the top or cover 62, as shown by FIG. 1. The tube 32 has an end 32b which may reside inside an enclosure or chamber 60a, inside the discharge receptacle 60. The discharge receptacle 60 may be a jar made of glass or plastic. The discharge or mucus 70 taken or sucked out of an infant's nose is shown at the bottom of the receptacle 60.

The tube 46 has a first end 46a which is attached to an end 50b of the nozzle 50. The nozzle 50 has a portion 52 at which the nozzle 50 is attached to the tube 46. The nozzle 50 also has a portion 54 which has a decreasing outer diameter towards an end 50a. The decreasing diameter allows the end 50a and the nozzle 50 to penetrate further into a nostril of a small child or infant. The tube 46 has a second end 46b which is attached to an end 42a of the tube 42. The tube 42 may be a stiff plastic tube which acts as an air line. The tube 42 may be inserted through a hole 62b in the top or cover 62, as shown by FIG. 1. The tube 42 has an end 42b which may reside inside an enclosure or chamber 60a, inside the discharge receptacle 60.

The end 32b of the tube 32 may lie a distance D1, which may be one to two inches, above the bottom inside surface 60b inside the enclosure 60a of the jar 60. The end 42b of the tube 42 may lie a distance D2, which may be one-half to one inch, above the bottom inside surface 60b. Both the end 42b and the end 32b should be higher than the discharge level of discharge or mucus 70 in receptacle 60. The end 42b of the suction tube 42 will typically be slightly closer to the bottom inside surface 60b than the end 32b of the vacuum tube 32 in order to keep constant vacuum flow.

Typically, the body portion 27 of the vacuum device 20 would include a battery compartment. Typically batteries would be used for powering the vacuum device 20 and the vacuum device 20, and generally apparatus 10, would be portable. However, the vacuum device 20 could be powered in any other known way such as by being plugged into an electrical outlet.

The vacuum device 20 could be rechargeable. The discharge receptacle 60 may be capable of holding one to two ounces of fluid. The top or cover 62 may be a screw top and the discharge receptacle 60 may have threads near its top 60c, for screwing and tightening the cover 62 onto the receptacle 60. The tubes 30 and 46 may be surgical tubes. The nozzle 50 may be a surgical rubber nose tip. The nozzle 50 may have differing outer or inner diameters, such as for example, ranging from one-half inch to two inches.

The tube 46 may be fifteen to eighteen inches long. The tube 30 may be four to six inches long. Each of the tubes 30 and 46 may have an inner diameter of one sixteenth to one eighth of an inch. The cover 62 and the receptacle 60 form an airtight enclosure 62a within the receptacle 60, with the exception of the holes 62a and 62b through which the tubes 32 and 42, respectively, pass.

In operation, a parent, for example, would turn on the power for the vacuum device 20 by, for example, moving the switch 28 towards the “ON” position. When the vacuum device 20 is powered it will cause air to be sucked into the tube 32, with a suction force, in the direction U1, up through end 32b. The air flow continues through the tube 32 and then into and through the tube 30 in the direction U2. The airflow passes through vacuum connection 24 and into the vacuum device 20. The suction and the airflow in the directions U1 and U2 created by the vacuum device 20 causes air to flow and suction to occur in the direction F1 into the end 50a of the nozzle 50. The air flows into and through the nozzle 50 and into end 46a of the tube 46. The air flows in the direction F2 through the tube 46, out the end 46b and into the end 42a of the tube 42. The air then flows through the tube 42 in the direction F3, and out the end 42b of the tube 42.

With the vacuum device 20 turned “ON”, the parent inserts the end 50a of the nozzle 50 into a nostril of an infant or a child. Mucus within the nostril of the child is sucked out, into nozzle 50 through nozzle end 50a. The mucus flows through nozzle 50, through end 50b, and through tube 46 in the direction of F2. The mucus then continues to flow through end 46b and into end 42a and through tube 42 in the direction F3. The mucus then flows out of end 42b of tube 42 and settles on the bottom surface 60b of the receptacle 60, as accumulated mucus 70 shown in FIG. 1.

The end 32b of the tube 32 is further above the bottom surface 60b of the receptacle 60 than the end 42b of the tube 42, to avoid the mucus 70 from being sucked into the tube 32, or the tube 30, or the vacuum device 20.

The apparatus 10 and the vacuum device 20 may be small hand held units which are powered by battery or plug-in or both. The apparatus 10 and the vacuum device 20 may gently sucks mucus from a baby's or small child's nose. The nozzle 50 can be disposable so that it can be taken off and replaced by another similar nozzle. The receptacle 60 may be taken off the attachment device 27 and cleaned.

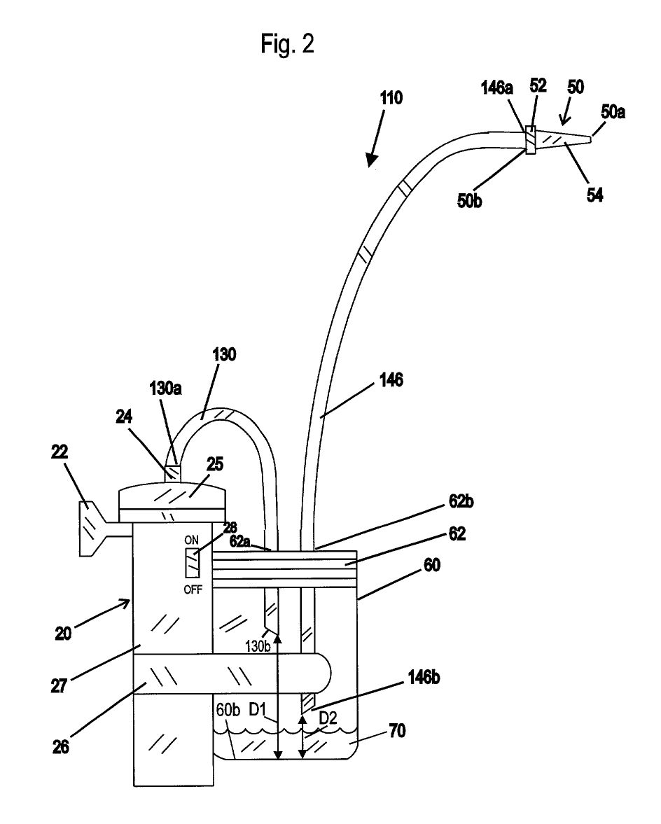

Instead of having tubes 32 and 42 inserted through cover 62, the tubes 30 and 46 can be inserted through the openings 62a and 62b, respectively, of the cover 62. In that case, the tubes 32 and 42 could be eliminated, as shown by FIG. 2. FIG. 2 shows an apparatus 110 in accordance with a second embodiment of the present invention. FIG. 2 is the same as FIG. 1 except that tubes 130 and 146 (having ends 130a and 130b and 146a and 146b, respectively) have replaced tubes 30 and 46, respectively, tubes 32 and 42 have been eliminated, and tubes 130 and 146 have been inserted through the openings 60a and 60b, respectively.

The apparatus 10 and the vacuum device 20 may be devices which can be held in a person's hand. In particular the vacuum device 20 may be a hand held unit.

As an alternative to the embodiment shown in FIG. 1, the nozzle 50 may be attached directly to the vacuum device 20. For example, nozzle end 50b may be attached to vacuum connection 24 without the use of the other components shown in FIG. 1. In this embodiment the one or more of the tubes 30, 32, 42, and 46 and the receptacle 60 may be eliminated. The nozzle 50 may also be considered to be a type of tube in accordance with the present invention. A discharge unit similar to receptacle 60, may be located within the vacuum device 20 and may receive the mucus discharge. The nozzle 50 tip end 50a may vary in size for various applications. The nozzle 50, which can be also called a nostril tip, could be plastic with a throw-away sterile covering or made out of replaceable surgical rubber to be cleaned and reused or thrown away.

The vacuum device 20 may be powered through a power cord which may be connected to a cigarette lighter in an automobile. In this manner the vacuum device 20 and the apparatus 10 could be used while driving or could be recharged through the cigarette lighter of an automobile.

As shown in FIG. 1, nozzle or tip 50 may be made of plastic or surgical rubber. But the portion 52 near end 50b may be shaped larger to prevent a person from inserting the nozzle 50 into the nose too far. The portion 52 may have a diameter larger than the average nostril for which it is intended for, i.e. if intended for use with infants, the portion 52 would have a diameter large enough to prevent the nozzle 50 from being inserted too far into the nostril, i.e. the portion 52 will contact the outer surface of the nostril and will not be able to be inserted into the nostril. The portion 52 may in this manner act like a stopper or ring. The diameter of portion 52 should be substantially larger than the largest diameter of portion 54.

Although the invention has been described by reference to particular illustrative embodiments thereof, many changes and modifications of the invention may become apparent to those skilled in the art without departing from the spirit and scope of the invention. It is therefore intended to include within this patent all such changes and modifications as may reasonably and properly be included within the scope of the present invention's contribution to the art.

1. An apparatus comprising
a vacuum device; and
a first tube having a first end and a second end connected to the vacuum device;
wherein when the vacuum device is powered there is a suction force created at the first end of the first tube;
and wherein the first tube is adapted to suck mucus out from a nostril of a nose of a person through the first end of the first tube.

2. The apparatus of claim 1 further comprising
a nozzle having a first end and a second end, the first end connected to the first end of the first tube; and
wherein the second end of the nozzle is inserted into the nostril and the nose of the person in order to suck mucus out from the nostril of the nose of the person.

3. The apparatus of claim 1 further comprising
a second tube having a first end and a second end;
a receptacle;
wherein the second end of the first tube is connected to the receptacle, so that the suction force created by the vacuum device causes mucus to be deposited into the receptacle;
wherein the first end of the second tube is connected to the receptacle;
and wherein the second end of the second tube is connected to the vacuum device.

4. The apparatus of claim 3 further comprising
an attachment device which attaches the receptacle to the vacuum device.

5. The apparatus of claim 3 further comprising
a cover which can be attached to the receptacle;
wherein the cover has a first opening and a second opening and wherein the first tube passes through the first opening and the second tube passes through the second opening; and
wherein the cover together with the receptacle forms an enclosure which is airtight except for the first and second openings in the cover.

6. The apparatus of claim 3 further comprising
a cover which can be attached to the receptacle;
wherein the cover has a first opening and a second opening;
wherein the cover together with the receptacle forms an enclosure which is airtight except for the first and second openings in the cover;
further comprising a third tube having a first end and a second end;
and a fourth tube having a first end and a second end;
wherein the first end of the third tube is connected to the second end of the first tube;
wherein the third tube is inserted into the first opening of the cover and the second end of the third tube lies in the enclosure;
wherein the first end of the fourth tube is connected to the first end of the second tube;
and wherein the fourth tube is inserted into the second opening of the cover and the second end of the fourth tube lies in the enclosure.

7. The apparatus of claim 6
wherein the receptacle has a bottom surface;
wherein the second end of the third tube is fixed at a first location; and
wherein the second end of the fourth tube is fixed at a second location which is farther from the bottom surface of the receptacle than the first location.

8. The apparatus of claim 5
wherein the receptacle has a bottom surface;
wherein the second end of the first tube is fixed at a first location; and
wherein the first end of the second tube is fixed at a second location which is farther from the bottom surface of the receptacle than the first location.

9. A method comprising the steps of:
sucking mucus out from a nostril of a nose of a person by the use of a vacuum device.

10. The method of claim 9 wherein
the vacuum device is connected to a first tube and mucus is sucked out from the nostril of the nose of the person through the first tube.

11. The method of claim 10 wherein
the first tube is connected to a nozzle and the mucus is sucked out from the nostril of the nose of the person through the nozzle.

12. The method of claim 10 wherein
the first tube has first and second ends and the second end of the first tube is connected to a receptacle;
and further comprising sucking mucus out from the nostril of the nose through the first tube and depositing the mucus into the receptacle.

13. The method of claim 12 wherein
the vacuum device is connected to a second end of a second tube, with a first end of the second tube connected to the receptacle; and
wherein a suction force through the second tube causes mucus to be deposited in the receptacle.

14. The method of claim 12 further comprising
attaching the receptacle to the vacuum device.

15. The method of claim 12 further comprising
attaching a cover to the receptacle; and
wherein the cover has a first opening and wherein the first tube passes through the first opening.

16. The method of claim 15 wherein
the cover has a second opening and wherein the second tube passes through the second opening;
and wherein the cover together with the receptacle forms an enclosure which is airtight except for the first and second openings in the cover.

17. The method of claim 13 further comprising
attaching a cover to the receptacle, the cover and the receptacle forming an enclosure;
connecting a first end of a third tube to the second end of the first tube;
inserting the third tube into a first opening of the cover so that a second end of the third tube lies in the enclosure;
connecting a first end of a fourth tube to the first end of the second tube; and
inserting the fourth tube into a second opening of the cover so that a second end of the fourth tube lies in the enclosure.

18. The method of claim 17 wherein
wherein the receptacle has a bottom surface;
wherein the second end of the third tube is fixed at a first location; and
wherein the second end of the fourth tube is fixed at a second location which is farther from the bottom surface of the receptacle than the first location.
